(19) United States
(12) Patent Application Publication
(10) Pub. No.: US 2005/0148528 A1
(43) Pub. Date: Jul. 7, 2005

(54) Method for reducing platelet count

(75) Inventor: Steven T. Gately, Palatine, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NEOPHARM, INC, LAKE FOREST, IL (US)

(21) Appl. No.: 10/514,970

(22) PCT Filed: May 19, 2003
(86) PCT No.: PCT/US03/15922

Related U.S. Application Data
(60) Provisional application No. 60/382,411, filed on May 20, 2002.

(51) Int. Cl. A61K 48/00; A61K 9/127; C12N 15/88

(52) U.S. Cl. 514/44; 424/450; 435/458

(57) ABSTRACT

The invention provides a method for treating elevated platelet levels in patients using oligonucleotides in formulations that enhance penetration of the oligonucleotide into cells. Certain oligonucleotides found suitable for use in this method are anitisense oligonucleotides. Of the antisense oligonucleotide that are effective, it has been found that antisense oligonucleotides that inhibit the expression of the raf-1 gene can be used, for example, formulated in liposomes. The method has the advantage that the formulation of oligonucleotide can be administered to human patients and the platelet count will decrease in the absence of additional therapeutic treatment steps, including other chemotherapeutic or radiation treatments.

### CROSS REFERENCE TO RELATED APPLICATIONS

This application claims priority to U.S. Provisional Patent Application 60/382,411, filed May 20, 2002.

### FIELD OF THE INVENTION

This invention pertains to the use of oligonucleotides to reduce the platelet count in patients with elevated platelet counts. The oligonucleotides are formulated with agents that improve cell penetration.

### BACKGROUND OF THE INVENTION

Essential thrombocytosis is a nonreactive, chronic, myeloproliferative disorder that is associated with sustained megakaryocyte proliferation that results in a platelet increase. The disease is characterized by a platelet count greater than 600,000/mm³, megakaryocytic hyperplasia, splenomegaly, and a clinical course complicated by hemorrhagic and/or thrombotic episodes. Unpleasant effects of the disease include headaches, pain caused by microvascular occlusion of the toes and fingers, gangrene, and/or erythromelalgia (burning pain). Approximately 6,000 cases are diagnosed each year, however, there has been speculation that it may be much more prevalent. Although uncommon, death can occur from thrombotic complications. Patients afflicted with this disease are susceptible to contracting acute myeloid leukemia, which occurs in 1-5% of the patient population.

Treatment for the disease usually involves the administration of the antimetabolites hydroxyurea or anagrelide. Both treatments have side effects. The former is relatively inexpensive but carries a risk of secondary malignancy and gastrointestinal tract complications causing about 30% of the patient population to cease using the drug. Anagrelide causes fluid retention.

Presently there exists a need for new methods for treating essential thrombocytosis in human patients. The invention provides such a method. These and other advantages of the invention, as well as additional inventive features, will be apparent from the description of the invention provided herein.

### BRIEF SUMMARY OF THE INVENTION

The invention provides a method for treating elevated platelet levels in patients using oligonucleotides in formulations that enhance penetration of the oligonucleotide into cells. Certain oligonucleotides found suitable for use in this method are antisense oligonucleotides. Of the antisense oligonucleotides that are effective, it has been found that antisense oligonucleotides that inhibit the expression of the raf-1 gene can be used. The oligonucleotides can be formulated in cationic liposomes, which have net positive charges under the conditions of use. The method has the advantage that the formulation of oligonucleotide can be administered to human patients and the platelet count will decrease in the absence of additional therapeutic treatment steps, including other chemotherapeutic or radiation treatments.

### DETAILED DESCRIPTION OF THE INVENTION

The invention provides a method for reducing the platelet count in patients using formulations of oligonucleotides that include an agent that enhances penetration of the oligonucleotide into cells. In other words, the invention pertains to the use of an oligonucleotide to prepare a medicament for reducing the platelet count in a patient characterized in that said oligonucleotide is formulated with an agent that enhances penetration of the oligonucleotide into cells. The agent can be a liposome forming material, a lipophilic chemical modification of the oligonucleotide or any material that aids the oligonucleotide in penetrating a cell.

Suitable patients are those patients that have elevated platelet counts, such patients include individuals diagnosed with essential thrombocytosis.

Any oligonucleotide can be used in the present invention so long as it reduces the platelet count when administered to patients. Oligonucleotides that have been found useful in the method include antisense oligonucleotides. Antisense oligonucleotides that inhibit the expression of the Raf-1 gene have been identified as one suitable group of oligonucleotides. In particular, the oligonucleotide having SEQ ID No. 1 (5′-GTGCTCCATTGATGC-3′) is suitable for the method, and other antisense oligonucleotide inhibitors of the Raf-1 gene are known in the art (see, e.g., international patent publication WO94/15645).

The oligonucleotides can be prepared by any suitable method. For example, oligonucleotides can be synthesized using beta-cyanoethyl phosphoramidite chemistry on a Biosearch 8750 DNA synthesizer. The oligonucleotide can be modified to stabilize the oligonucleotide. For example, as is known, phosphorothioate groups, among other groups, can be added using 3H-1,2-benzodithiole-3-1,1,1-dioxide as a sulfurizing agent during oligonucleotide synthesis.

Oligonucleotides can be purified following their synthesis by any suitable technique. For example, reverse phase HPLC chromatography columns and polyacrylamide gels can be used.

To determine the quality and integrity of an oligonucleotide a small aliquot can be ³²P-end-labeled and visualized by polyacrylamide gel electrophoresis (20% acrylamide and 5% bisacrylamide) followed by densitometric scanning of the labeled products. Alternatively, oligonucleotide integrity can be verified by monitoring the absorbance at 280 nm after high pressure liquid chromatography such as on a reverse phase column.

Cationic liposomes can be prepared with any suitable cationic lipid that is not significantly toxic to humans in the quantities that are required to be administered. Exemplary cationic lipids include 1,2-dioleoyl-3-trimethyl ammonium propane (DOTAP), 1,2-dimyristoyl-3-trimethyl ammonium propanes (DMTAP), and dimethyldioctadecyl ammonium bromide (DDAB), all of which are commercially available from Avanti Polar Lipids (Alabaster, Ala., USA).

Suitable relative molar amounts of cationic lipid:phosphatidyl choline:cholesterol are in the range of about 0.1-25:1-99:0-50. More preferably, relative molar amounts range from about 0.2-10:2-50:1-25, still more preferably about 0.5-5:4-25:2-15, and still more preferably the amounts range from about 0.75-2:5-15:4-10. In one method liposomes were prepared using a cationic lipid along with phosphatidylcholine and cholesterol in a molar ratio of 1:3.2:1.6.

Liposomal formulations also contain suitable amounts of antioxidants such as α-tocopherol or other suitable antioxidants. Suitable amounts range from about 0.001 or more to about 5 wt. % or less.

In liposome formulations any ratio of oligonucleotide to lipid that provides for incorporation of the majority of the oligonucleotide can be used. For example, a mass ratio of between about 1:100 and 1:2 can be used. A ratio of between about 1:50 to 1:3 or 1:30 to 1:10 can be used. The preferred ratio is about 1:15 oligonucleotide:lipid by mass.

Liposomes can be prepared by any suitable method. For example, the lipids can be dissolved in a nonpolar solvent such as chloroform or methanol, and evaporated to dryness in a round-bottomed flask using a rotary vacuum evaporator. The dried lipid film can be hydrated overnight at 4° C. by adding 1 ml of oligonucleotide at 1.0 mg/ml in phosphate-buffered saline (PBS). The film can be dispersed by vigorous vortexing and the liposome suspension sonicated for 5 min in a bath type sonicator (Laboratory Supplies, Hicksville, N.Y., USA). The unencapsulated oligonucleotide can be removed by washing the liposomes and centrifugation three times at 75,000×g for 30 min in phosphate buffered saline.

To determine the efficiency of oligonucleotide encapsulation an aliquot of the preparation containing ³²P-end-labeled oligonucleotide can be counted in a scintillation counter. The liposome-encapsulated oligonucleotide can be stored at 4° C. and used within 2 weeks of preparation.

The method provides for the human administration of pharmaceutical preparations which in addition to liposome formulations of active agents include non-toxic, inert pharmaceutically suitable excipients. Pharmaceutically suitable excipients include solid, semi-solid or liquid diluents, fillers and formulation auxiliaries of all kinds. The invention also includes pharmaceutical preparations in dosage units. This means that the preparations are in the form of individual parts, for example vials, syringes, capsules, pills, suppositories, or ampoules, of which the content of the liposome formulation of active agent corresponds to a fraction or a multiple of an individual dose. The dosage units can contain, for example, 1, 2, 3, or 4 individual doses, or ½, ⅓, or ¼ of an individual dose. An individual dose preferably contains the amount of active agent which is given in one administration and which usually corresponds to a whole, a half, a third, or a quarter of a daily dose.

Although the formulations can be administered locally, orally, parenterally, intraperitoneally, and/or rectally, intravenous administration is preferred.

The following examples further illustrate the invention but, of course, should not be construed as in any way limiting its scope.

### EXAMPLE 1

This example demonstrates the preparation of liposomes containing an oligonucleotide suitable for the treatment of essential thrombocytosis.

Dimethyldioctadecylammonium Bromide (DDAB), egg phosphatidylcholine and cholesterol were purchased from Avanti Polar Lipids (Alabaster Ala.). Antisense oligonucleotide SEQ ID No. 1 (5′-GTG CTC CAT TGA TGC-3′), which is directed toward the translation initiation site of human c-raf-1 mRNA, was purchased from Hybridon Inc. (Milford. Mass.).

Oligonucleotide containing cationic liposomes were prepared using DDAB, phosphatidyl choline and cholesterol in a molar ratio of about 1:3.2:1.6. The lipids (5 mg DDAB, 20 mg phosphatidyl choline and 5 mg cholesterol) were dissolved in 2 ml chloroform and evaporated to dryness at 37° C. using a vacuum evaporator. Liposome-encapsulated oligonucleotide SEQ ID No. 1 was prepared by hydrating the dried lipid film overnight at 4° C. with 1 ml of oligonucleotide solution at 2.0 mg/ml in normal saline. The film was dispersed by vigorous vortexing and the liposome suspension was sonicated for 10 min in a bath type sonicator (Model XL 2020, M. isonix Inc., Farmingdale, N.Y.). The liposome encapsulated oligonucleotide was stored at 4° C. and was used within 3 days after preparation.

The encapsulation efficiency of oligonucleotide in liposomes was determined by adding ³²P end-labeled oligonucleotide to excess of unlabeled oligonucleotide prior to its formulation in liposomes. The unencapsulated oligonucleotide was removed by ultracentrifugation of the liposome solution at 100,000 g for 20 min followed by washing the liposomes twice in normal saline and recentrifuging. The oligonucleotide encapsulation efficiency was determined by scintillation counting of an aliquot of the preparation. The encapsulation of AS-oligonucleotide into liposomes prepared using DDAB, phosphatidylcholine and cholesterol by conventional film method (Method 1) was found to be 88.0±2.0% (n=2).

### EXAMPLE 2

This example demonstrates the preparation of liposomes containing an oligonucleotide suitable for the treatment of essential thrombocytosis.

Lipids (5 mg DDAB, 20 mg phosphatidylcholine, 5 mg cholesterol and 0.3 mg α-tocopherol) were dissolved in 4 ml t-butanol, filtered through a 0.22μ filter and lyophilized. The lyophilized lipids were reconstituted at room temperature with 2.0 mg/ml oligonucleotide in normal saline at an oligonucleotide to lipid mass ratio of 1:15 and vortexed vigorously for 2 min. The vials were then hydrated at room temperature for 2 h. At the end of hydration, vials were sonicated for 10 min in a bath type sonicator (Model XL 2020, Model XL 2020, Misonix Inc. Farmingdale, N.Y.). Blank liposomes were prepared exactly as described above in the absence of oligonucleotide. The liposome encapsulated oligonucleotide was stored at 4° C. and was used within 3 days after preparation.

The encapsulation efficiency of oligonucleotide in liposomes was determined by adding ³²P end labeled oligonucleotide to excess of unlabeled oligonucleotide prior to its formulation in liposomes. The unencapsulated oligonucleotide was removed by ultracentrifugation of the liposome solution at 100,000 g for 20 min followed by washing the liposomes twice in normal saline and recentrifuging. The oligonucleotide encapsulation efficiency was determined by scintillation counting of an aliquot of the preparation. The encapsulation efficiency of liposomes prepared by this method was 87.2±2.5%.

The encapsulation efficiencies of oligonucleotide in cationic liposomes were similar regardless of whether the liposomes were prepared using dimethyldioctadecylammonium bromide, phosphatidylcholine and cholesterol by conventional film method as in Example 1 or lyophilization of lipid methods as in this example. Approximately 10% of the oligonucleotide remained free. Nevertheless, trace amounts of free oligonucleotide are not expected to interfere with patient health, efficacy, clinical observations, or data analysis. Accordingly, for studies described below, unencapsulated oligonucleotide was not removed from the formulations.

### EXAMPLE 3

The following example demonstrates that the administration of a liposomal formulation of oligonucleotide can be used to dramatically reduce human platelet counts in a sustained manner.

Cancer patients with recurrent malignancies for whom palliative radiotherapy was indicated were chosen for treatment. Liposome-encapsulated oligonucleotide was prepared as described in Example 2 and administered by intravenous infusion over 30 minutes. Ten doses were given daily for five days per week for two weeks. Two hours after administration, external beam radiotherapy was administered to a total dose of 30 Gy in 10 fractions. The daily dose of LErafAON was escalated in cohorts of 36 patients, doses were about 1, 2, 4, and 6 mg/kg/day. Blood samples were obtained for pharmacokinetic analysis.

Patients chosen for this study had histologically-confirmed malignancy which has recurred or progressed after initial definitive treatment and for which no standard therapy is available. In such patients radiation therapy was indicated for the disease and site of the disease. More than four weeks since any prior therapy, with recovery from any side effects. Patients had measurable or evaluable tumors documented 1-2 weeks prior to study entry, performance status (ECOG) 0-2, age at least 18 years, and adequate organ function.

Patients were excluded if they were receiving any concurrent antitumor therapy or if they had a history of excessive toxicity from prior radiation therapy. Patients were excluded if they had any infection requiring parenteral antibiotics, HIV infection, or seropositivity for Hepatitis B or Hepatitis C. Patients who were pregnant or nursing were excluded. Lastly, any patient having a central nervous system metastasis was excluded.

At each time point 5 cc of blood was drawn, labeled with patient initials and time of sample, permitted to clot, and centrifuged to separate the serum. Serum was transferred to a screw top tube, carefully labeled with patient initials and accession number, date and time of sampling and frozen at −70° C. Samples were drawn 30 min prior to infusion (baseline) and in the last minute of the infusion post infusion. In the Tables that follow the numbers in column “N” represent the number of patients studied at each dosage. Tables 1, 2, 3, and 4 provide the data for 1, 2, 4, and 6 mg/kg doses. The numbers in the Mean and Median columns represent the mean and median platelet counts in the patients investigated.

TABLE 1

| Timepoint | N | Mean | Median |
|---|---|---|---|
| Baseline | 4 | 258.8 | 286.5 |
| Wk 1 Pre Infusion | 4 | 260.8 | 298.0 |
| Wk 1 Post Infusion | 3 | 196.7 | 235.0 |
| Wk 2 Pre Infusion | 3 | 206.7 | 238.0 |
| Wk 3 Pre Infusion | 3 | 210.3 | 246.0 |
| Wk 4 Pre Infusion | 2 | 174.0 | 174.0 |
| Wk 5 Pre Infusion | 1 | 110.0 | 110.0 |
| Wk 6 Pre Infusion | 1 | 113.0 | 113.0 |
| Wk 7 Pre Infusion | 1 | 88.0 | 88.0 |
| Wk 8 Pre Infusion | 1 | 89.0 | 89.0 |
| Wk 8 Post Infusion | 1 | 88.0 | 88.0 |
| LOCF | 4 | 199.0 | 231.0 |

Table 1 demonstrates that administration of about 1 mg of oligonucleotide in a liposomal formulation is sufficient to substantially reduce platelet counts with each administration when the dose is administered weekly for 8 weeks.

TABLE 2

| Timepoint | N | Mean | Median |
|---|---|---|---|
| Baseline | 3 | 331.0 | 333.0 |
| Wk 1 Pre Infusion | 3 | 316.7 | 291.0 |
| Wk 1 Post Infusion | 2 | 290.5 | 290.5 |
| Wk 2 Pre Infusion | 3 | 299.3 | 278.0 |
| Wk 2 Post Infusion | 1 | 193.0 | 193.0 |
| Wk 3 Pre Infusion | 3 | 275.3 | 274.0 |
| Wk 4 Pre Infusion | 3 | 282.0 | 300.0 |
| Wk 4 Post Infusion | 2 | 250.5 | 250.5 |
| Wk 5 Pre Infusion | 2 | 247.5 | 247.5 |
| Wk 6 Pre Infusion | 2 | 238.0 | 238.0 |
| Wk 6 Post Infusion | 2 | 222.5 | 222.5 |
| Wk 7 Pre Infusion | 2 | 227.0 | 227.0 |
| Wk 8 Pre Infusion | 2 | 234.5 | 234.5 |
| Wk 8 Post Infusion | 1 | 259.0 | 259.0 |
| LOCF | 3 | 260.7 | 259.0 |

Table 2 demonstrates that administration of about 2 mg of oligonucleotide in a liposomal formulation is sufficient to substantially reduce platelet counts with each administration when the dose is administered weekly for 8 weeks.

TABLE 3

| Timepoint | N | Mean | Median |
|---|---|---|---|
| Baseline | 4 | 349.3 | 329.0 |
| Wk 1 Pre Infusion | 4 | 337.0 | 311.0 |
| Wk 1 Post Infusion | 4 | 254.5 | 251.5 |
| Wk 2 Pre Infusion | 4 | 280.8 | 273.5 |
| Wk 2 Post Infusion | 1 | 219.0 | 219.0 |
| Wk 3 Pre Infusion | 3 | 191.7 | 179.0 |
| Wk 4 Pre Infusion | 3 | 155.0 | 169.0 |
| Wk 4 Post Infusion | 1 | 119.0 | 119.0 |
| Wk 5 Pre Infusion | 3 | 132.0 | 102.0 |
| Wk 6 Pre Infusion | 3 | 145.0 | 103.0 |
| Wk 7 Pre Infusion | 3 | 118.7 | 115.0 |
| Wk 8 Pre Infusion | 2 | 120.5 | 120.5 |
| Wk 8 Post Infusion | 1 | 81.0 | 81.0 |
| LOCF | 4 | 134.5 | 119.0 |

Table 3 demonstrates that administration of about 4 mg of oligonucleotide in a liposomal formulation is sufficient to substantially reduce platelet counts with each administration when the dose is administered weekly for 8 weeks.

TABLE 4

| Timepoint | N | Mean | Median |
|---|---|---|---|
| Baseline | 7 | 306.3 | 308.0 |
| Wk 1 Pre Infusion | 8 | 279.6 | 287.0 |
| Wk 1 Post Infusion | 3 | 198.7 | 203.0 |
| Wk 2 Pre Infusion | 6 | 209.0 | 221.0 |
| Wk 2 Post Infusion | 4 | 177.5 | 190.0 |
| Wk 3 Pre Infusion | 6 | 158.3 | 141.5 |
| Wk 4 Pre Infusion | 5 | 100.2 | 98.0 |
| Wk 4 Post Infusion | 4 | 66.3 | 64.5 |
| Wk 5 Pre Infusion | 3 | 109.0 | 101.0 |
| Wk 6 Pre Infusion | 3 | 116.7 | 122.0 |
| Wk 6 Post Infusion | 1 | 68.0 | 68.0 |
| Wk 7 Pre Infusion | 3 | 94.7 | 115.0 |
| Wk 8 Pre Infusion | 3 | 90.0 | 86.0 |
| Wk 8 Post Infusion | 1 | 46.0 | 46.0 |
| LOCF | 8 | 139.3 | 92.0 |

Table 4 demonstrates that administration of about 6 mg of oligonucleotide in a liposomal formulation is sufficient to substantially reduce platelet counts with each administration when the dose is administered weekly for 8 weeks.

The present study shows that a liposomal oligonucleotide formulation can reduce platelet counts in a dose dependent manner. A dose of 6 mg of the oligonucleotide provided a reduction in platelets of about 85%. A dose of 4 mg of the oligonucleotide reduced the platelet count by about 75%. A dose of 2 mg was used to reduce the platelet count by about 35%. A dose of 1 mg was used to reduce the platelet count by about 60%.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for reducing the platelet count in a patient, comprising preparing a formulation of an oligonucleotide with an agent that enhances penetration of the oligonucleotide into cells and administering the formulation to a patient with an elevated platelet count:

2. The method of claim 1 wherein the agent that enhances penetration of the oligonucleotide into cells is a liposome-forming agent.

3. The method of claim 2, wherein the liposome-forming agent is cationic.

4. The method of claim 3 wherein the liposome-forming agent is 1,2-dioleoyl-3-trimethyl ammonium propane (DOTAP).

5. The method of claim 3, wherein the liposome-forming agent is 1,2-dimyristoyl-3-trimethyl ammonium propane propanes (DMTAP).

6. The method of claim 3 wherein the liposome-forming agent is dimethyldioctadecyl ammonium bromide (DDAB).

7. The method of claim 1, wherein the oligonucleotide is an antisense oligonucleotide.

8. The method of claim 7, wherein the antisense oligonucleotide is directed to Raf-1 mRNA.

9. The method of claim 1, wherein the administration is intravenous administration.

10. The method of claim 1, wherein the patient is a human patient.

11. (canceled)

12. (canceled)

13. (canceled)

14. (canceled)

15. (canceled)

16. (canceled)

17. (canceled)

18. (canceled)

19. (canceled)

20. A method of preparing a medicament for reducing the platelet count in a patient, the method comprising the formulation of an oligonucleotide with an agent that enhances penetration of the oligonucleotide into cells.

21. The method of claim 20, wherein the agent that enhances penetration of the oligonucleotide into cells is a liposome-forming agent.

22. The method of claim 21, wherein the liposome-forming agent is cationic.

23. The method of claim 22, wherein the liposome-forming agent is 1,2-dioleoyl-3-trimethyl ammonium propane (DOTAP).

24. The method of claim 22, wherein the liposome-forming agent is 1,2-dimyristoyl-3-trimethyl ammonium propane (DMTAP).

25. The method of claim 22, wherein the liposome-forming agent is dimethyldioctadecyl ammonium bromide (DDAB).

26. The method of claim 20, wherein the oligonucleotide is an antisense oligonucleotide.

27. The method of claim 26, wherein the antisense oligonucleotide is directed to Raf-1 mRNA.

28. The method of claim 20, wherein the formulation is suitable for intravenous administration.


SEQUENCE LISTING (1 sequence)

SEQ ID NO: 1 - DNA, 15 bp, Artificial
  Synthetic
gtgctccattgatgc